(19) United States
(12) Patent Application Publication
(10) Pub. No.: US 2002/0160947 A1
(43) Pub. Date: Oct. 31, 2002

(54) Synergistic method for prolonging allograft survival

(75) Inventors: Peter J. Elliott, Marlborough, MA (US); Wayne W. Hancock, Philadelphia, PA (US)

Correspondence Address: HALE AND DORR, LLP, 60 STATE STREET, BOSTON, MA, 02109

(73) Assignee: Millennium Pharmaceuticals, Inc., Cambridge, MA

(21) Appl. No.: 10/114,602

(22) Filed: Apr. 2, 2002

Related U.S. Application Data
(60) Provisional application No. 60/281,088, filed on Apr. 3, 2001.
(60) Provisional application No. 60/282,535, filed on Apr. 9, 2001.

(51) Int. Cl. A61K 38/13; A61K 31/407; A61K 31/4745

(52) U.S. Cl. 514/9; 514/291; 514/412

(57) ABSTRACT

The invention relates to allograft transplantation. More particularly, the invention relates to prolonging the survival of transplanted allografts. The invention provides a new method for improving allograft survival in a mammal. The method according to the invention provides a synergistic effect between lactacystin or lactacystin analogs and immunosuppressive drugs to prolong the survival of transplanted allografts in a mammal.

### CROSS-REFERENCE TO RELATED APPLICATIONS

This application claims priority from U.S. Provisional Patent Application Serial No. 60/281,088, filed on Apr. 3, 2001, and U.S. Provisional Patent Application Serial No. 60/282,535, filed on Apr. 9, 2001, the contents of which are hereby incorporated by reference in their entirety.

### BACKGROUND OF THE INVENTION

1. Field of the Invention

The invention relates to allograft transplantation. More particularly, the invention relates to prolonging the survival of transplanted allografts.

2. Summary of the Related Art

The proteasome is a large, multi-component protease central to progression of the cell cycle, activation of transcription, antigen processing and other crucial cellular processes. O. Coux, K. Tanaka, and A. L. Goldberg, Annu. Rev. Biochem. 65: 801-847 (1996), and A. L. Goldberg, Science 268: 522-523 (1995), teach that proteasome-mediated degradation of ubiquitinated and phosphorylated I-kappa-B-alpha is a key step in activation of NF-kappa-B. K. Tanaka et al., Adv. Immunol. 64: 1-38 (1997), teaches that NF-kappa-B activation is critical to the so-called “immunoproteasome” which is induced by IFN-gamma and is involved in immune responses. S. T. Smiley et al., Transplantation 70: 415-419 (2000), teaches that the immunoproteosome is involved in the rejection of transplanted allografts. W. W. Hancock et al., J. Immunol. 138: 164-170 (1987), teaches that activated T cells are required for allograft rejection. Wu et al., WO99/22729, teaches that inhibition of proteasome activity using lactacystin results in induction of apoptosis of activated T-cells, but not resting T cells, and suggests that proteasome inhibitors may be combined with immunosuppressive drugs to prevent graft rejection. Unfortunately, this has never been demonstrated in vivo, where the complexity of the immune response renders such a result highly unpredictable.

There is, therefore, a need for new methods to improve survival of transplanted allografts in a mammal. Ideally, such methods could provide true synergy between proteasome inhibitors and other immunosuppressive drugs.

### BRIEF SUMMARY OF THE INVENTION

The invention relates to allograft transplantation. More particularly, the invention relates to prolonging the survival of transplanted allografts. The invention provides a new method for improving allograft survival in a mammal. The method according to the invention provides a synergistic effect between lactacystin or lactacystin analogs and immunosuppressive drugs to prolong the survival of transplanted allografts in a mammal.

In the method according to the invention, a mammal bearing an allograft is administered a compound having the structure:

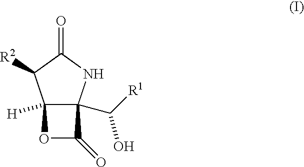

wherein R¹ is selected from the group consisting of C1-C6 alkyl, C3-C6 cycloalkyl, phenyl and phenyl(C1-C4)alkyl; and R² is selected from the group consisting of methyl, ethyl, n-propyl, n-butyl and iso-butyl; and the mammal is also administered an immunosuppressive drug, such as cyclosporine A, rapamycin, FK506, mycophenolate, corticosteroids, deoxyspergualin, or brequinar.

In the method according to the invention, even a normally sub-therapeutic dosage of an immunosuppressive drug can act synergistically with compound (I) to greatly prolong allograft survival in a mammal. The method according to the invention is useful for improving survival in patients who have received allograft transplantation. The method is also useful for studying the mechanism of allograft rejection.

### BRIEF DESCRIPTION OF THE DRAWINGS

FIG. 1 shows allograft survival times in control animals and in animals treated with cyclosporin A, compound (II), or compound (II) plus cyclosporin A.

FIG. 2 shows inhibition of proteasome function in hearts and blood of control animals and in animals treated with cyclosporin A or compound (II).

FIG. 3 shows a Western blot of I-kappa-B from normal heart, allograft, and allograft from animals treated with cyclosporin A or compound (II).

FIG. 4 shows graft histology from animals treated with vehicle or compound (II).

FIG. 5 shows results of RNase protection assays for various cytokines, chemokines and chemokine receptors from allografts from animals treated with vehicle or compound (II).

### DETAILED DESCRIPTION OF THE PREFERRED EMBODIMENTS

The invention relates to allograft transplantation. More particularly, the invention relates to prolonging the survival of transplanted allografts. The invention provides a new method for improving allograft survival in a mammal. The method according to the invention provides a synergistic effect between lactacystin or lactacystin analogs and immunosuppressive drugs to prolong the survival of transplanted allografts in a mammal.

The patents and publications cited herein reflect the knowledge in the art and are hereby incorporated by reference in entirety. Any inconsistency between these patents and publications and the present disclosure shall be resolved in favor of the present disclosure.

In the method according to the invention, a mammal bearing an allograft is administered a compound having the structure:

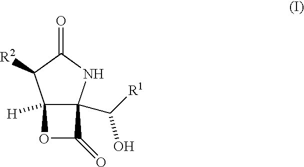

wherein R¹ is selected from the group consisting of C1-C6 alkyl, C3-C6 cycloalkyl, phenyl and phenyl(C1-C4)alkyl; and R² is selected from the group consisting of methyl, ethyl, n-propyl, n-butyl and iso-butyl; and the mammal is also administered an immunosuppressive drug, such as cyclosporine A, rapamycin or FK506, mycophenolate, corticosteroids, deoxyspergualin, brequinar.

In certain preferred embodiments R¹ is selected from the group consisting of C1-C6 alkyl. In a particularly preferred embodiment, R¹ is isopropyl.

In certain preferred embodiments, R² is preferably selected from the group consisting of ethyl, n-propyl, n-butyl and iso-butyl. In a particularly preferred embodiment, R² is n-propyl.

In one particularly preferred embodiment, in the lactacystin analog of formula (I) R¹ is isopropyl and R² is n-propyl, thus having the structure:

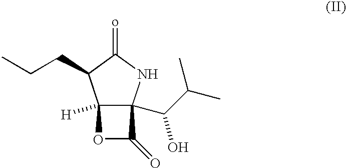

Synthesis of lactacystin and lactacystin lactones useful in the method according to the invention is taught in detail in U.S. Pat. No. 6,133,308, which is hereby incorporated by reference in its entirety.

Amounts and regimens for the administration of compounds (I) or (II) can be determined readily by those with ordinary skill in the clinical art. A desired dosage can be administered in one or more applications to obtain the desired results. Pharmaceutical compositions containing these compounds can be provided in unit dosage forms. Preferably, the compounds are administered parenterally, i.e., intravenously, subcutaneously, or intramuscularly. Typically, the compounds are administered to mammals, e.g. humans, by intravenous or subcutaneous injection at a dose of about 0.01 to about 10 mg/kg, preferably about 0.025 to about 1 mg/kg.

Compounds (I) or (II) may be formulated in any pharmaceutically acceptable carrier, excipient or diluent in which the compound is stable. Preferably, the formulation contains a water-miscible alcohol such as ethanol, isopropanol, or polyethylene glycol. Particularly preferred formulations include, without limitation, 45% isopropanol, 5% ethanol, 0.1% citric acid, and saline; and 50% polyethylene glycol.

Immunosuppressive drugs may be used in the method of the invention at dosages and regimens as recommended by the manufacturer.

Compounds (I) or (II) may be administered at the same time as the immunosuppressive drug or before or after the immunosuppressive drug, or may be administered in overlapping regimens with the immunosuppressive drug.

The following examples are intended to further illustrate certain particularly preferred embodiments of the invention and are not intended to limit the scope of the invention.

### EXAMPLE 1

### Effect of Compound (II) and/or Cyclosporine A on Graft Survival

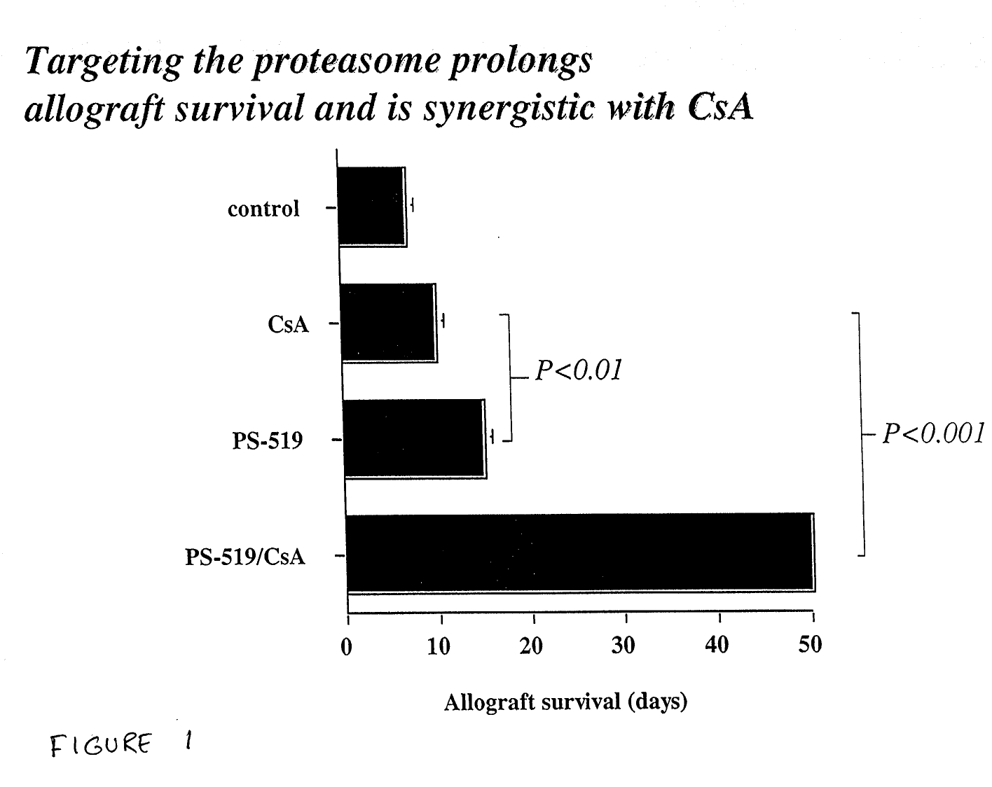

A conventional murine heterotopic vascularized cardiograft model (BALB/c to B6) (W. W. Hancock et al., Proc. Natl. Acad. Sci.(USA) 93:13967-13972 (1996)) was used to assess the effect of compound (II) on allograft survival. Allografted mice were injected intramuscularly with compound (II) (or vehicle) using 1 mg/kg/day. Cyclosporine A was administered for the first 14 days post-transplant at a dose of 10 mg/kg/day. The results are shown in FIG. 1. Vehicle controls rejected the allograft at about 7 days. Cyclosporine A alone prolonged allograft survival by only 3 days over the vehicle control. Compound (II) alone doubled survival time. Interestingly, when the dose of cyclosporine A was continued only 14 days, but the dose of compound (II) was continued daily until the end of the experiment, the allograft survived for 50 days. These results demonstrate that compound (II) can provide synergy with an immunosuppressive drug.

### EXAMPLE 2

### Effect of Compound (II) and/or Cyclosporine A on Proteasome Function In Vivo

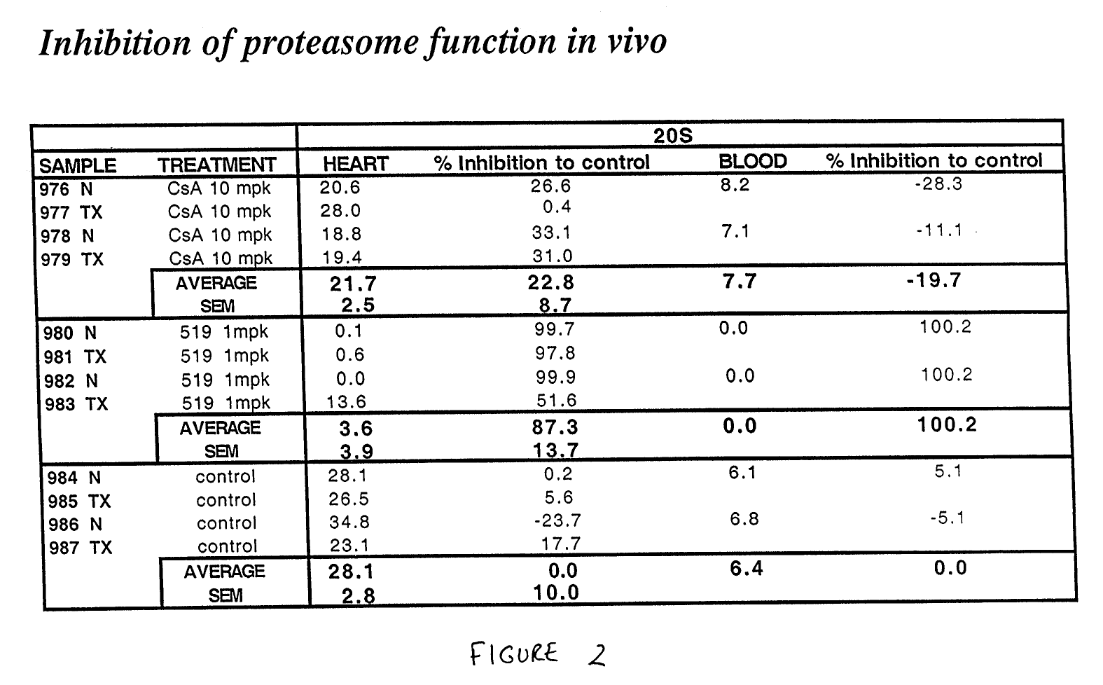

Proteasome activity in blood and heart of animals treated according to example 1 was measured as described in PCT publication WO 00/23614. The results are shown in FIG. 2. These results demonstrate that compound (II) strongly inhibits proteasome function in vivo, whereas cyclosporine A has a much weaker inhibitory effect.

### EXAMPLE 3

### Effect of Compound (II) and/or Cyclosporine A on Proteasome-Mediated I-kappa-B Degradation In Vivo

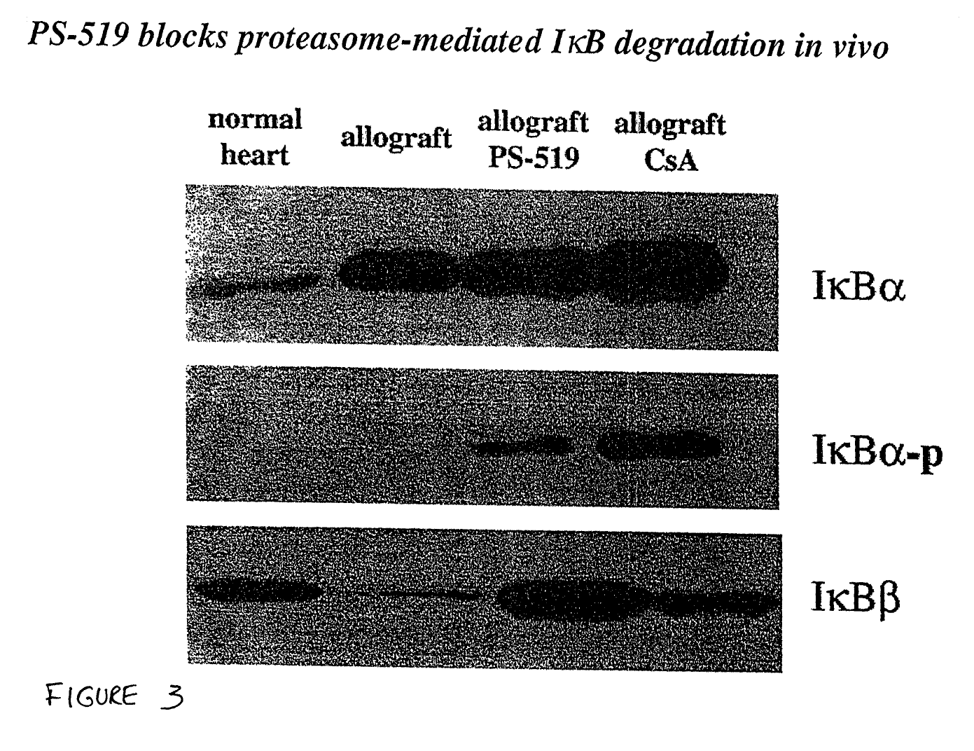

Western blotting was performed according to standard procedures (see e.g., Palombella et al., Cell 78: 773-785 (1994)) to determine whether compound (II) or cyclosporine A could inhibit proteasome-mediated degradation of I-kappa-B-alpha or I-kappa-B-beta from the allograft in animals treated according to Example 1. The results are shown in FIG. 3. These results demonstrate that both compound (II) and cyclosporine A could inhibit such proteasome-mediated degradation.

### EXAMPLE 4

### Effect of Compound (II) on Mononuclear Cell Infiltration and Graft Necrosis

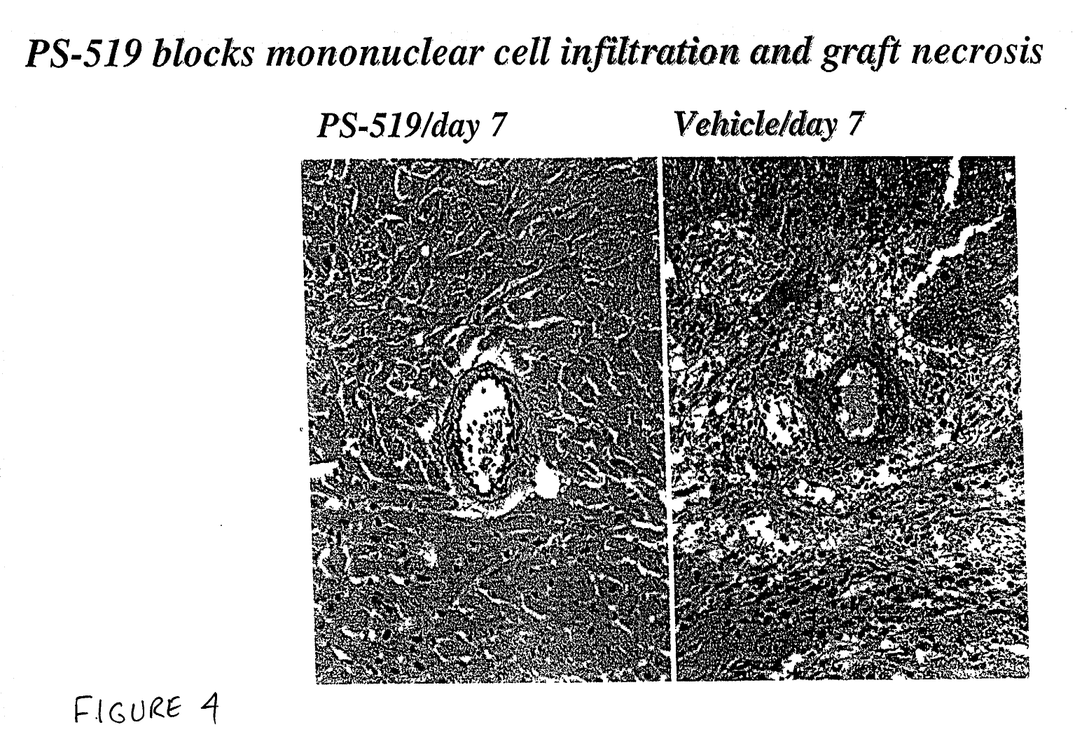

Grafts were taken from animals treated according to Example 1 for 7 days and subjected to standard histologic studies as follows. Grafts were fixed in formalin, embedded in paraffin, and paraffin sections were stained with hematoxylin and eosin (H & E) (W. W. Hancock et al., Proc. Natl Acad. Sci(USA) 93: 13967-13972 (1996)). The results are shown in FIG. 4. Compared to the acute rejection in vehicle treated animals, marked by myocyte necrosis and mononuclear cell infiltration, grafts from animals treated with compound (II) were almost normal.

### EXAMPLE 5

### Effect of Compound (II) on Intagraft Immune Activation

Grafts were taken from animals treated according to Example 1 for 7 days and subjected to RNase protection assays as follows:

Ribonuclease Protection Assay (RPA) of Cytokine, Chemokine and Chemokine Receptor Expression

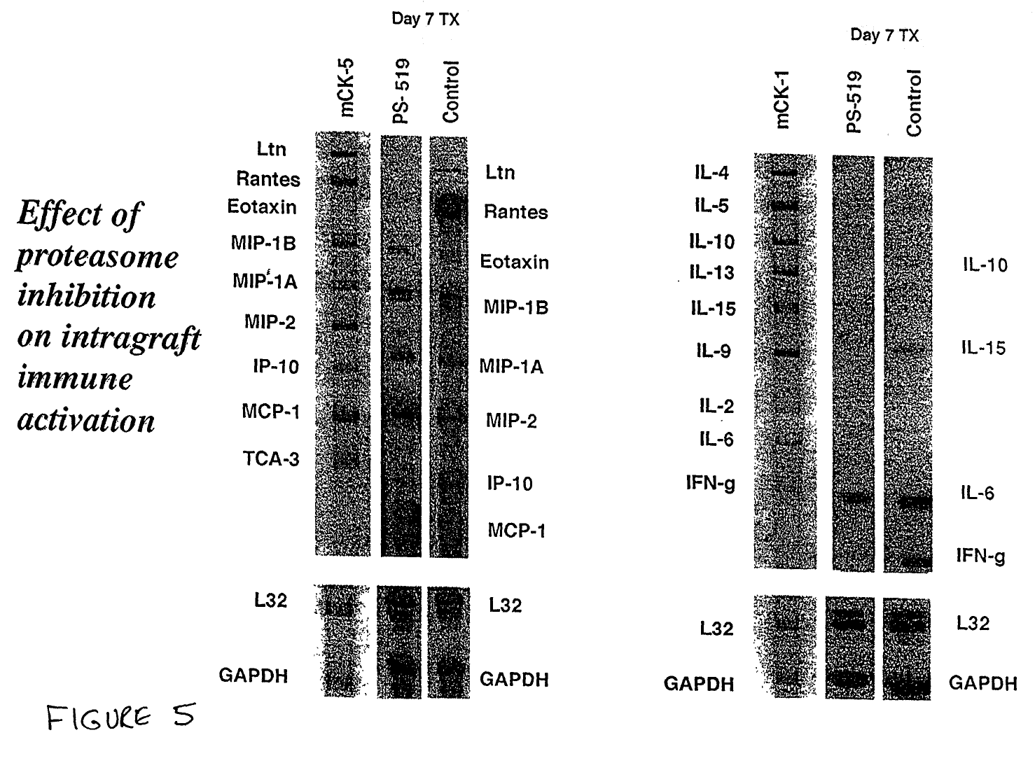

Cardiac RNA was isolated in guanidine-thiocyanate, with 2 rounds of acid phenol/chloroform extraction and alcohol precipitation (W. Gao et al., J. Clin. Investig. 105: 35-44 (2000)). RNA integrity was confirmed by agarose gel electrophoresis and quantitated by optical density measurement (260 nm). RNA from each mouse was evaluated using the Riboquant system (Pharmingen); mouse template sets mCK5 and mCK3b were used for detection of chemokines and cytokines, respectively, and template sets mCR5 and mCR6 were used to detect CC and CXC chemokine receptors. In addition, a riboprobe for mouse CXCR3 was prepared in-house. In vitro transcription was carried out in transcription buffer supplemented with [a32P] UTP (3000 Ci/mmol; Amersham Life Science, Arlington Heights, Ill.) and T7 RNA polymerase. After DNaseI treatment, the riboprobe was isolated by phenol/chloroform extraction and ammonium acetate/ethanol precipitation, and labeling efficiency was determined by measuring Cherenkov activity in a scintillation counter. Each riboprobe set was diluted to the optimal activity defined by the manufacturer, added to 20 μg of kidney RNA, heated to 90° C., allowed to cool to 56° C.; and annealed overnight. After RNase and proteinase K treatment, protected RNA hybrids were purified by phenol/chloroform extraction and ammonium acetate/ethanol precipitation and separated by electrophoresis on 5% polyacrylamide/8 M urea gels. Gels were dried, subjected to autoradiography using Kodak Biomax MS2 film (Eastman Kodak, Rochester, N.Y.), and autoradiographs were scanned into Adobe Photoshop (Adobe, San Jose, Calif.). RNA bands were quantitated by densitometric analysis with NIH Image (NIH, Bethesda, Md.), and results were normalized for L32 and GAPDH gene expression The results are shown in FIG. 5. These results demonstrate that compound (II) inhibited intragraft expression of key cytokines (IFN-gamma, IL-10), chemokines (IP-10 RANTES) and chemokine receptors (CXCR3).

What is claimed is:

1. A method for prolonging allograft survival in a mammal bearing an allograft, the method comprising administering a compound having the structure: 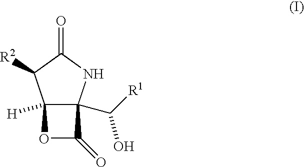
wherein R¹ is selected from the group consisting of C1-C6 alky, C3-C6 cycloalkyl, phenyl and phenyl(C1-C4)alkyl; and R² is selected from the group consisting of methyl, ethyl, n-propyl, n-butyl and iso-butyl; and further comprising administering to the mammal an immunosuppressive drug.

2. The method according to claim 1, wherein the immunosuppressive drug is selected from the group consisting of cyclosporine A, rapamycin and FK506.

3. The method according to claim 1, wherein R¹ is selected from the group consisting of C1-C6 alkyl.

4. The method according to claim 3, wherein R¹ is isopropyl.

5. The method according to claim 1, wherein R² is selected from the group consisting of ethyl, n-propyl, n-butyl and iso-butyl.

6. The method according to claim 1, wherein R² is n-propyl.

7. The method according to claim 1 wherein compound (I) is injected subcutaneously.

8. The method according to claim 1 wherein compound (I) is injected intravenously.

9. The method according to claim 8, wherein compound (I) is administered at a dose of about 0.025 to about 1 mg/kg.

10. A method for prolonging allograft survival in a mammal bearing an allograft, the method comprising administering a compound having the structure: 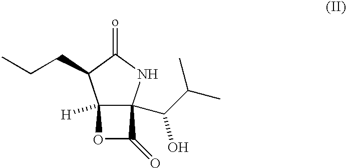
and further comprising administering to the mammal an immunosuppressive drug.

11. The method according to claim 10 wherein compound (II) is injected subcutaneously.

12. The method according to claim 10 wherein compound (II) is injected intravenously.

13. The method according to claim 12, wherein compound (II) is administered at a dose of about 0.025 to about 1 mg/kg.
